(19) United States
(12) Patent Application Publication
(10) Pub. No.: US 2012/0064049 A1
(43) Pub. Date: Mar. 15, 2012

(54) USE OF STEM CELLS FROM HAIR ROOT SHEATHS AND KERATINOCYTE PRECURSOR CELLS FOR THE REGENERATION OF AGED SKIN

(75) Inventor: Thomas Hunziker, Oberhofen (CH)

(73) Assignee: SKINREPHAIR LTD., Zurich (CH)

(21) Appl. No.: 13/321,385

(22) PCT Filed: May 28, 2010
(86) PCT No.: PCT/EP2010/003248

§ 371 Date: Nov. 18, 2011

(30) Foreign Application Priority Data
Jun. 5, 2009 (EP) 09007450.1

(51) Int. Cl. A61K 8/98; C12N 5/071; A61P 17/00; A61P 17/02; A61Q 19/08; A61K 35/12

(52) U.S. Cl. 424/93.7; 435/325

(57) ABSTRACT

The present invention relates to the use of stem cells from hair root sheaths and/or keratinocyte precursor cells for the regeneration of aged but otherwise healthy and non-injured skin for the cosmetic purposes and for the prevention of skin diseases. In addition, the invention is directed to a cosmetic method for the regeneration of aged skin.

The present invention relates to the use of stem cells from hair root sheaths and/or keratinocyte precursor cells for the regeneration of aged but otherwise healthy and non-injured skin for cosmetic purposes and for the prevention of skin diseases. In addition, the invention is directed to a cosmetic method for the regeneration of aged skin.

These days a young beautiful skin has increasingly gained a psychological-social relevance, and for this reason a large number of topical preparations are marketed that are supposed to counter or reverse skin aging. However, a reasoned medical scientific proof of any lasting effects is missing for the most part.

On one hand, skin aging depends on age with great individual variability (intrinsic or chronological aging), and on the other hand depends on external damaging factors, in particular cumulative sun (UV)-exposure, malnutrition, drug abuse, mechanical wear and also nicotine abuse (extrinsic aging).

An aged skin manifests itself in a thinning (atrophy) of the epidermis as well as the dermis, increased wrinkle formation and loss of elasticity (elastosis of the dermis), dry skin and loss of turgor as well as possibly irregularities in pigmentation (e.g. lentigines solares) of the skin. The life-long regeneration (homeostasis) of the skin is on one hand based on the continuous replacement of the epidermis by division of the stem cells of the keratinocytes in the lowest layer (stratum basale) with subsequent differentiation into horn discs when advancing to the outer layer, the horny layer (stratum corneum).

In aged skin this process is disordered, thereby leading to atrophy. If the differentiation of the keratinocytes is strongly disfunctional, degeneration can become possible, i.e. the development of so-called white skin cancer (spinalioma, basalioma) or cancer precursors (actinic keratoses). This is mainly the case in chronically sun-exposed skin regions, which underlines the triggering relevance of UV-radiation. A sustainable therapeutic approach for the regeneration of aged skin is therefore desirable not only for cosmetic reasons. A fully functional epidermis is also important for the preservation of the structure and function of the underlying dermis because the cell systems of these two compartments of the skin (keratinocytes of the epidermis and fibroblasts or vascular endothelium of the dermis) communicate with each other via soluble factors (e.g. cytokines, growth factors). Hence, a fully functional epidermis can also contribute to the regeneration of age-related changes of the dermis, i.e. the dermal atrophy with elastosis and vascular dilatation (teleangiectasia).

The important keratinocyte precursor- or stem cells, which are decisive for the homeostasis of the epidermis, are localized in the epithelial hair root sheaths (outer root sheath: ORS), i.e. in the lower dermis and, thus, protected form harmful UV-radiation (the particularly harmful UV-B only barely penetrates through the epidermis). These ORS cells can be isolated from plucked scalp hair in the growth phase and can also be propagated in the laboratory.

Repigmentation of skin is known as an application of ORS stem cells or melanocyte precursur cells derived therefrom from autologous hair root sheaths (Vanscheidt & Hunziker, Dermatology, 904, 2009, WO 2009/049734).

Furthermore, chronic wounds are treated with precursor cells for epidermal keratinocytes from autologous hair root sheaths (EpiDex®, Euroderm; Tausche et al., Wound Repair and Regeneration, 11(4), 248-252, 2003, EP 1 198 557 B1, EP 1 326 654 B1).

Next to the treatment of chronic wounds, the treatment of other skin defects with keratinocyte precursor cells is disclosed, e.g. after the surgical removal of skin tumours or tattoos or due to injuries, etc. (EP 1 198 557 B1, EP 1 702 979 A1).

Epidermal neural crest stem cells (EPI-NCSC) are multipotent stem cells, that are derived from the embryonal neural crest and which are located in hair root sheaths. These stem cells permanently renew themselves and are capable of differentiating into all important cell derivatives of the neural crest including neurons, nerve-supporting cells, smooth muscle cells, bone/cartilage cells and melanocytes. These stem cells can even produce cell types that are typical for the mesoderm. In a mouse model for backbone injuries it was shown that these stem cells can fuse with adult skeletal muscle fibers, that the introduced nuclei are functional and that adult skeletal muscle is a suitable environment for the long-term survival of the stem cell nuclei (Sieber-Blum & Hu, Stem Cell Rev., 4(4), 256-60, 2008). From their investigations on skeletal muscle in mice the authors conclude that such pluripotent stem cells could provide attractive properties for future cell replacement therapies and/or biomedical developments.

It is the objective of the present invention to regenerate aged but otherwise healthy and non-injured skin.

This objective is solved by using stem cells from hair root sheaths and/or keratinocyte precursor cells for the regeneration of aged but otherwise healthy and non-injured skin.

The term “healthy and non-injured skin” describes skin without defects, in particular without wounds as well as without inflammatory, infectious or degenerative skin diseases, benign or malignant skin tumours or their precursors (e.g. actinic keratosis, lentigo maligna), post-operative skin transformations such as after skin transplantations etc.

The term aged skin in the context of the invention relates to intrinsically and/or extrinsically aged skin preferably having at least one of the following aging symptoms: thinning (atrophy), fine wrinkle formation, loss of elasticity (elastosis), increased vulnerability with a tendency for hemorrhage after low level trauma as well as possibly irregularities in pigmentation, but also having deep wrinkles or crinkles, with dry, possibly scaling/keratotic skin surface, decreased activity of sebaceous glands, decreased skin turgor, decreased skin fat content, a tendency toward cracks and pseudoscars, dilatation of small blood vessels (teleangiectasia), loss of the ability to regenerate and a wound healing dysfunction connected thereto. These appearances are based on the disordered activity of transcription factors or tumour suppressor genes (e.g. NF-kappa B, c-Myc, p53), which regulate the proliferation and differentiation of skin cells (in particular of the keratinocytes and fibroblasts) and thereby the homeostasis of the skin. For example, it was possible to show for NF-kappa B in older mice that its blockage elicited a biologically younger skin condition within two weeks for a limited time period.

The regeneration of aged but otherwise healthy and non-injured skin according to the invention also has medical advantages in addition to cosmetic aspects. The keratinocyte precursor cells introduced into the skin do not only thicken the epidermis as fully differentiated keratinocytes but also divide regularly and thereby, as time goes by, replace “old” cells that divide more slowly as well as degenerated cells. Moreover, they positively influence cells in their direct environment (fibroblasts, vascular endothelial cells, melanocytes) by the excretion of cytokines and growth factors, which as a whole improves the health and aging condition of the skin in the long run, whereby the wrinkle formation is reduced (regeneration of collagen fibers), the elasticity is increased (regeneration of elastic fibers) and possibly present irregularities in pigmentation (regulation of the melanin production and distribution) decline. By influencing the microcirculation of the skin the nutritive situation of the skin is improved, which, e.g. also increases the trophic situation and the activity of sebaceous glands and thereby normalizes the oil condition of the skin. Therefore, the term regeneration of aged but otherwise healthy and non-injured skin according to the invention not only encompasses the cosmetic regeneration but also the medical prophylactic regeneration of skin.

In a preferred embodiment the present invention relates to an exclusively cosmetic use of stem cells from hair root sheaths and/or keratinocyte precursor cells for the regeneration of aged but otherwise healthy and non-injured human and mammalian skin.

In a further preferred embodiment the present invention is directed to the use of stem cells from hair root sheaths and/or keratinocyte precursor cells for the prevention of skin diseases in humans and mammals, preferably of skin diseases selected from the group consisting of skin diseases based on genetic or acquired dysfunctions of skin cohesion, skin homeostasis, skin differentiation and/or skin barrier function, preferably epidermolysis bullosa, xeroderma pigmentosum, progeria, ichthyosis, status after surgical intervention and after x-ray irradiation and exogenously induced atrophy or hyperplasia, in particular hyperplastic scars and keloids, preferably atrophy induced by topic or systemic corticosteroids. Therefore, the invention also relates to the use of said cells for the preparation of a medicament for the prevention of skin diseases.

Autologous as well as allogenic and xenogenic cells can be used, whereas autologous cells are preferred for avoiding immune reactions.

In a further aspect, the invention is directed to a cosmetic method for the regeneration of aged but otherwise healthy and non-injured skin, comprising the step of applying stem cells from hair root sheaths and/or keratinocyte precursor cells on aged but otherwise healthy and non-injured skin.

For the treatment of aged skin preferably stem cells from hair root sheaths of the patient him- or herself or preferably keratinocyte precursor cells derived therefrom, so called autologous keratinocyte precursor cells, are employed. The keratinocyte precursor cells from hair root sheaths contain pluripotent stem cells, which regenerate the epithelial skin structures all life long. For this reason they also have a high proliferation potential even for older donors. These stem cells or precursor cells cannot only be prepared non-invasively but also repeatedly from any scarcely haired donor and without a skin biopsy. Hence, the application of these cells allows for a mostly non-invasive possibility to regenerate aged skin sustainably, in particular the epidermis.

The stem cells from hair root sheaths and/or keratinocyte precursor cells as well as their preparation, isolation, propagation and storage is sufficiently known to those skilled in the field of dermatology. Preferably, the stem cells and/or keratinocyte precursor cells for practicing the present invention are prepared from hair root sheaths, in particular the outer epithelial hair root sheath in the growth phase (anagenic phase).

The preparation of these cells is quite simple, painless and does not bear any health risks. For example, the hairs used for preparing the cells can be obtained by plucking terminal hair, in particular of anagenic hair of the kapillitum. It is of advantage when the hair stems from the person or the mammal to be treated.

Preferably, the term preparation also comprises the at least partial isolation and/or enrichment as well as the propagation or selection of cells in the culture. In a preferred embodiment the cells are enzymatically released, e.g. by means of a trypsin solution in a concentration of 0.01 to 10, preferably 0.025 to 0.1 and more preferred about 0.05% trypsin, optionally together with EDTA and preferably in PBS over preferably 5 to 60, more preferably 10 to 15 minutes at preferably about 20° C. or more preferred 37° C. from the hair root sheath of a removed hair. This type of enzymatic removal is particularly gentle for the cells to be prepared. Also, other enzyme systems such as e.g. dispase can be employed. Further possible steps for the preparation include the termination of the enzymatic release by means of the addition of serum and Ca/Mg, the centrifugation of the suspension and the suspending/introduction of the cell-containing sediments in a solution suitable for the application on skin or a biodegradable or non-biodegradable carrier matrix. A preferred application is the suspending of the cell-containing sediment in a thrombin-containing solution, which allows for an immediate fixation of the applied cells in a thin layer on skin pre-treated with fibrinogen, and therefore enables a homogenous non-occlusive application onto skin in any body region. Preferably, with the inventive method autologous stem cells from hair root sheaths and/or keratinocyte precursor cells are employed.

Optionally, the cells can be employed as suspension or sediment or optionally as a cell extract. When doing so, they can be introduced in a biocompatible solution or a biocompatible carrier.

The application step is preferably carried out by means of a suspension having 10² to 10⁹, more preferably having 10³ to 10⁵ cells/cm² per hair area to be treated, given, for example by means of a syringe or as a spray or in a biocompatible carrier or non-woven web. This can be carried out with a biocompatible solution (e.g. PBS, cell culture medium, thrombin) or by means of a biocompatible biological (e.g. fibrin, hyaluronan, collagen) or synthetic (e.g. polyurethane, carboxymethyl cellulose, polylysin, nanoparticle) carrier. The cells can be employed as vital proliferation-capable or growth-arrested cells (e.g. by means of treatment with mitomycin C or X-ray irradiation) or also as cell extracts (such as e.g. lyophilisates, sonicates, stimulates). In addition, cells derived from these cells under different incubation conditions in vitro (e.g. variable pO₂- and/or pCO₂ concentrations, variable culture media, variable matrix substrates, variable feed cells in direct cell contact or separated in two chamber culture systems, submersed or organotypical culture) can be employed.

The term application within the context of the invention encompasses the simple application of the cells onto the skin. Preferably, the cells are fixated to the skin for this purpose, e.g. by means of a fibrin adhesive, and are protected with a common occlusion bandage. However, any other suitable form of application is possible, e.g. by integrating the cells in biological or synthetic matrices and subsequent application of these. Furthermore, the cells can preferably be introduced directly into the skin. For this purpose the skin to be treated is physically and/or mechanically de-epidermised prior to the application, and this is preferably done by means of dermabrasio, superficial laser application (e.g. Fraxel re:pair® CO₂-laser, Soltamedical, USA), or superficial needle puncture (e.g. Dermaroller®, Skintes, CH), whereby planar or preferably punctual, i.e. grid-type fractional defects in the epidermis are produced, which allow for the penetration of the applied cells into the skin such as in a transepidermal application. By involving the upper dermis layers in this ablation process there is also a dermal stimulation and a dermal regeneration.

In a preferred embodiment of the method of the invention the epidermis is ablated physically and/or mechanically before the application of the cells by means of superficial dermabrasio, laser application and/or superficial needle puncture.

In a more preferred embodiment the epidermis is ablated before the application of the cells in the form of partial/fractional de-epidermisation with variable depth of penetration by means of a laser in fraxel modality and/or a dermaroller.

The stem cells from hair root sheaths and/or keratinocyte precursor cells are preferably present as enriched or isolated cells as an extract, suspension, solution, or comprised in a biocompatible biological or synthetic carrier during the application or introduction.

In a further preferred embodiment the stem cells from hair root sheaths and/or keratinocyte precursor cells can be propagated in vitro by culturing and/or be selected by means of specialised culturing conditions (e.g. variable pO₂- and/or pCO₂ concentrations, variable culture media, variable matrix substrates, variable feed cells in direct cell contact or separated in two chamber culture systems, submersed or organotypic culture, etc.) according to advantageous criteria such as, for example the content of stem cells and/or keratinocyte precursor cells, stem cell potential (mono- versus multi-/pluripotent), proliferation capacity, differentiation potential, survival capacity, etc. Selection in the context of the invention means the intentional selection of specific stem cells and/or keratinocyte precursor cells or accompanying cells having advantageous properties (no stem cells or keratinocyte precursor cells having functions such as interactive stimulation of the proliferation or differentiation of the stem cells and/or keratinocyte precursor cells in the context of feed cells).

The cell-treated skin is preferably covered occlusively after the application, in particular when the skin was mechanically pre-treated and still is partially “open”, in order to protect the skin but also the applied cells from drying, infections, and mechanical strain and in order to create an optimal environment for the growth of the cells.

In order to prevent immune reactions by autologous, in particular by allogenic or xenogenic cells, preferably at least one active agent inhibiting an (auto)immune reaction, preferably a topical or systemic corticosteroid and/or topical calcineurine-inhibitor is administered

It has further been found that the additional application of melanocyte precursor cells has an advantageous effect on the structure and physiology of aged skin. Therefore, in a preferred embodiment the method of the invention relates to additionally applying melanocyte precursor cells to the skin, in particular before, after or preferably simultaneous to the stem cells and/or keratinocyte precursor cells. It is most preferred not to separate the melanocyte precursor cells during the preparation of the stem cells from hair root sheaths and/or keratinocyte precursor cells.

The cells employed according to the invention can preferably comprise additional cells comprised in hair root sheaths, preferably other precursor cells next to stem cells and/or keratinocyte precursor cells and optionally melanocyte precursor cells.

Moreover, the present invention is directed to the above method for the prevention of skin diseases in mammals and humans with aged but otherwise healthy and non-injured skin, in particular for the prevention of skin diseases selected from the group consisting of skin diseases based on genetic or acquired dysfunctions of skin cohesion, skin homeostasis, skin differentiation and/or skin barrier function, preferably epidermolysis bullosa, xeroderma pigmentosum, progeria, ichthyosis, status after surgical intervention and after x-ray irradiation and exogenously induced atrophy or hyperplasia, in particular hyperplastic scars and keloids, preferably atrophy induced by topic or systemic corticosteroids. Preferred embodiments of this method are to be learned in analogy to the above-described cosmetic method.

The present invention will be illustrated by means of the following examplary application.

### EXAMPLE

From a 50 year old woman having strongly UV-aged facial skin featuring irregular epidermal atrophy, fine wrinkle formation, cheek teleangiectasia, dryness and reduced skin turgor 50 anagenic hairs were plucked from the scalp skin, the hair roots were separated and these were incubated for 10 minutes in trypsin solution (0.05% trypsin/0.02% EDTA in PBS without Ca/Mg) at 37° C. The microscopic control after inactivation of the trypsin by addition of PBS with Ca/Mg and 20% human AB serum confirmed the release of all cells of the epithelial hair root sheaths from the hair shaft. After passage through a cell sieve and subsequent centrifugation (1200 rpm, 10 min., at room temperature) the isolated cells were taken up in PBS with Ca/Mg and 5% glucose and drop-applied to the areas at the forehead and the cheeks (0.1 ml per 5 cm², corresponding to ˜5×10³ cells/cm²), which were pre-treated with the CO₂laser in fraxel modality (grid-like ablation of ˜20% of the treated epidermis area all the way to the upper dermis, penetration depth ˜100 μm). The treated area was subsequently covered occlusively with a modern wound bandage for a total of one week. The first bandage change was done after 3 days. Afterwards, the treated skin areas were treated in the morning with a sunscreen factor >50 and in the evening with a care lotion. Within 3 months there was an impresssive smoothing of the skin surface with a reduction of skin dryness, increase in turgor and homogenisation of the epidermal structure and also the cheek teleangiectasia was slightly regressing, and taken as a whole with a very satisfying cosmetic result for the patient.

1. Use of stem cells from hair root sheaths and/or keratinocyte precursor cells for the regeneration of aged but otherwise healthy and non-injured skin.

2. Use according to claim 1 for the cosmetic treatment of human and mammalian skin.

3. Use according to claim 1 for the prevention of skin diseases in humans and mammals, preferably of skin diseases selected from the group consisting of skin diseases based on genetic or acquired dysfunctions of skin cohesion, skin homeostasis, skin differentiation and/or skin barrier function, preferably epidermolysis bullosa, xeroderma pigmentosum, progeria, ichthyosis, status after surgical intervention and after x-ray irradiation and exogenously induced atrophy or hyperplasia, in particular hyperplastic scars and keloids, preferably atrophy induced by topic or systemic corticosteroids.

4. Use according to claim 1 for the preparation of a medicament.

5. Cosmetic method for the regeneration of aged but otherwise healthy and non-injured skin, comprising the step of applying stem cells from hair root sheaths and/or keratinocyte precursor cells on aged but otherwise healthy and non-injured skin.

6. Method according to claim 5, wherein the stem cells from hair root sheaths and/or keratinocyte precursor cells are autologous cells.

7. Method according to claim 5, wherein the epidermis is physically and/or mechanically ablated before the application of the cells, preferably by means of superficial dermabrasio, laser application and/or superficial needle puncture.

8. Method according to claim 7, wherein the epidermis is ablated before the application of the cells in the form of partial/fractional de-epidermisation with variable depth of penetration by means of a laser in fraxel modality and/or a dermaroller.

9. Method according to claim 5, wherein the cells are in the form of enriched or isolated cells as extract, suspension, solution, or comprised in a biocompatible biological or synthetic carrier.

10. Method according to claim 5, wherein the stem cells from hair root sheaths and/or keratinocyte precursor cells are propagated by culturing in vitro and/or selected.

11. Method according to claim 5, wherein prior to, during and/or after the application of the cells at least one active agent inhibiting an (auto)immune reaction, preferably a topical or systemic corticosteroid and/or topical calcineurine-inhibitor is administered.

12. Method according to claim 5, wherein additionally melanocyte precursor cells are applied to the skin.

13. Method according to claim 5, wherein next to stem cells from hair root sheaths and/or keratinocyte precursor cells and optionally melanocyte precursor cells the cells comprise additional cells comprised in hair root sheaths, preferably other precursor cells.
